(19) United States
(12) Patent Application Publication
(10) Pub. No.: US 2009/0274627 A1
(43) Pub. Date: Nov. 5, 2009

(54) Composition for Regeneration of Periodontal Soft Tissue and Method for Producing the Same

(75) Inventors: Yoichi Yamada, Nagoya-shi (JP); Minoru Ueda, Nagoya-shi (JP); Kazuto Okabe, Nagoya-shi (JP); Tomoyuki Kohgo, Nagoya-shi (JP)

Correspondence Address: OLIFF & BERRIDGE, PLC, P.O. BOX 320850, ALEXANDRIA, VA, 22320-4850, US

(73) Assignee: National University Corporation Nagoya University, Nagoya-shi, Aichi (JP)

(21) Appl. No.: 12/226,488

(22) PCT Filed: Apr. 19, 2007
(86) PCT No.: PCT/JP2007/000428

§ 371 Date: Feb. 26, 2009

(30) Foreign Application Priority Data
Apr. 19, 2006 (JP) 2006-115932

(51) Int. Cl. A61K 49/00; A61K 35/12; A61P 19/04

(52) U.S. Cl. 424/9.2; 424/93.7; 424/93.72

(57) ABSTRACT

It is intended to provide a composition for regeneration of periodontal soft tissue less invasively and a method for producing the composition. The composition for regeneration of periodontal soft tissue is prepared so as to contain a cell selected from an undifferentiated/stem cell or a blast cell having an ability to form gingiva, a matrix material and blood platelet plasma. With the composition, free gingiva as well as attached gingiva can be regenerated less invasively with an effective and good aesthetics.

### TECHNICAL FIELD

The present invention relates to a composition that is used to regenerate soft periodontal tissue and to a method of producing this composition

### BACKGROUND

Various oral surgical treatments for periodontal diseases have been implemented in recent years. In periodontal diseases, a portion of the periodontal tissues may undergo recession or is lost, either in the affected region per se or due to a surgical treatment thereto. However, from a position of securing post-treatment functionality and maintaining aesthetics thereof, the needs for repair and regeneration of such periodontal tissues is ever growing.

Periodontal tissue is composed of hard tissue (e.g., the alveolar bone, which supports the tooth) and soft tissue (e.g., the gingiva). As shown in FIG. 1, the soft periodontal tissue comprises the attached gingiva which is affixed by alveolar bone, and the free gingiva, which is not affixed by this hard tissue.

With regard to the repair and regeneration of periodontal tissues, for example, regeneration methods utilizing grafting of cells, e.g., osteoblasts for the regeneration of alveolar bone and its junction regions that connect with the periodontical tissues have been disclosed (Japanese Patent Application Laid-open Nos. 2004-201612 and 2004-000497). Also known is a guided tissue regeneration (GTR) method, in which the regeneration of new connective tissue attachment, alveolar bone, connective tissue, and so forth is promoted by establishing a space between the gingiva and dental root for periodontal tissue regeneration to take place in. A technique that uses material that induces periodontal tissue regeneration (Emdogain (product name), from Seikagaku Corporation) is also known; this material contains enamel matrix proteins taken from the dental germ of immature pigs.

Further with regard to methods for repairing gingival tissue, technique that collects connective tissue fragments or epithelial tissue fragments from the patient's oral cavity or from other tissues, and grafts the same, or utilize the same to cover the root surface that has become exposed by suturing the gingiva.

Among the periodontal tissues, the repair and regeneration of soft tissue, such as the gingiva, is strongly desired from the aesthetic standpoint. More specifically, the gingival margin and the gingival interdental papilla strongly affect the aesthetics of the gums and dental alignment (FIG. 1). The gap formed by recession of the interdental papilla, known as the black triangle, in particular can significantly spoil the appearance of the gums and dental alignment. In addition to the recession caused by periodontal diseases, the aforementioned free gingiva may also undergo recession due to improper oral care. Natural regeneration of the free gingiva becomes difficult when recession of the free gingiva is left unattended beyond a certain time interval.

### DISCLOSURE OF THE INVENTION

Meanwhile, the goal in the patent documents 1 and 2 cited above is the regeneration of hard tissue, such as alveolar bone with its high priority from the functional standpoint, and the same also applies to the use of GTR as well as the use of the material that induces periodontal tissue regeneration. Thus, none of these techniques is able to bring about a satisfactory restoration of the gingival tissue. With regard to the aforementioned tissue fragment grafting procedure, satisfactory aesthetics had not always been obtained in addition to the problem of the invasiveness associated with acquisition of the tissue graft and its implantation.

In view of the preceding discussion, no investigation has been made as yet of a low-invasive method of regenerating periodontal soft tissue, nor has such been found. In addition, no investigation has been made of a method that also effectively regenerates the free gingiva, such as the interdental papilla, nor has such been found.

An object of the present invention is therefore to provide a composition for the low-invasive regeneration of periodontal soft tissue and a method of producing this composition. A further object of the present invention is to provide a grafting composition for the effective regeneration of free gingiva, such as the interdental papilla, and a method of producing this composition. A further object of the present invention is to provide a composition for the regeneration of highly aesthetic gums and a method of producing this composition.

The present inventors investigated the use of mesenchymal stem cells and fibroblasts for the regeneration of periodontal soft tissue, i.e., the attached gingiva and the free gingiva such as the gingival margin and the gingival interdental papilla that are regarded as difficult to regenerate, and have discovered that periodontal soft tissue could be effectively regenerated by injecting these cells together with a matrix material at the soft tissue site where regeneration is desired. The present invention was achieved based on this discovery. Thus, the present invention provides the following means capable of solving at least one of the problems cited above.

The present invention provides a periodontal soft tissue regenerating composition that comprises cells selected from mesenchymal stem cells and gingival fibroblasts, and a matrix material. The composition of the present invention may also contain a cell growth factor. The periodontal soft tissue for the composition of the present invention is preferably the attached gingiva and/or the free gingiva, and the free gingiva is preferably the gingival interdental papilla. The composition of the present invention can be for local injection into periodontal tissue. The periodontal soft tissue is also preferably the interdental papilla.

The composition of the present invention may contain, as its matrix material, one or more items selected from the following (a) to (d):

(a) hyaluronic acid and derivatives thereof

(b) collagen and derivatives thereof

(c) fibrin and fibrinogen

(d) plasma and platelets

The matrix material is preferably selected from at least the (a) hyaluronic acid and derivatives thereof. In this aspect, the cells are preferably the mesenchymal stem cells. The matrix material is also preferably selected from at least the (b) collagen and derivatives thereof. In this aspect, the cells are preferably gingival fibroblasts.

The matrix material may also be preferably selected from at least the (d) plasma and platelets. Further, the matrix material is also preferably selected from each of the (a) hyaluronic acid and derivatives thereof and the (d) plasma and platelets. In this aspect, the cells may be mesenchymal stem cells. The matrix material is also preferably selected from each of the (b) collagen and derivatives thereof and the (d) plasma and platelets. The matrix material from the (d) plasma and platelets can be platelet-rich plasma.

The present invention may also provide a method of producing the periodontal soft tissue regenerating composition that comprises the steps of:

culturing cells selected from mesenchymal stem cells and gingival fibroblasts; and

mixing a matrix material with the cultured mesenchymal stem cells and/or gingival fibroblasts.

The cells in the inventive production method may be mesenchymal stem cells or gingival fibroblasts. The periodontal soft tissue may preferably be the attached gingiva or the free gingiva. The periodontal soft tissue may also be preferably the interdental papilla.

The above-referenced mixing step preferably uses, as the matrix material, one or more selected from the following (a) to (d):

(a) hyaluronic acid and derivatives thereof

(b) collagen and derivatives thereof

(c) fibrin and fibrinogen

(d) plasma and platelets

The mixing step may also use one or more items selected from at least the (a) hyaluronic acid and derivatives thereof as the matrix material. Furthermore, the mixing step may also use gingival fibroblasts as the cells and one or more selected from at least the (b) collagen and derivatives thereof as the matrix material. Moreover, the mixing step may also use as the matrix material at least platelet-rich plasma for the (d) plasma and platelets.

### BRIEF DESCRIPTION OF THE DRAWINGS

FIG. 1 is a schematic diagram of the gingival tissue region;

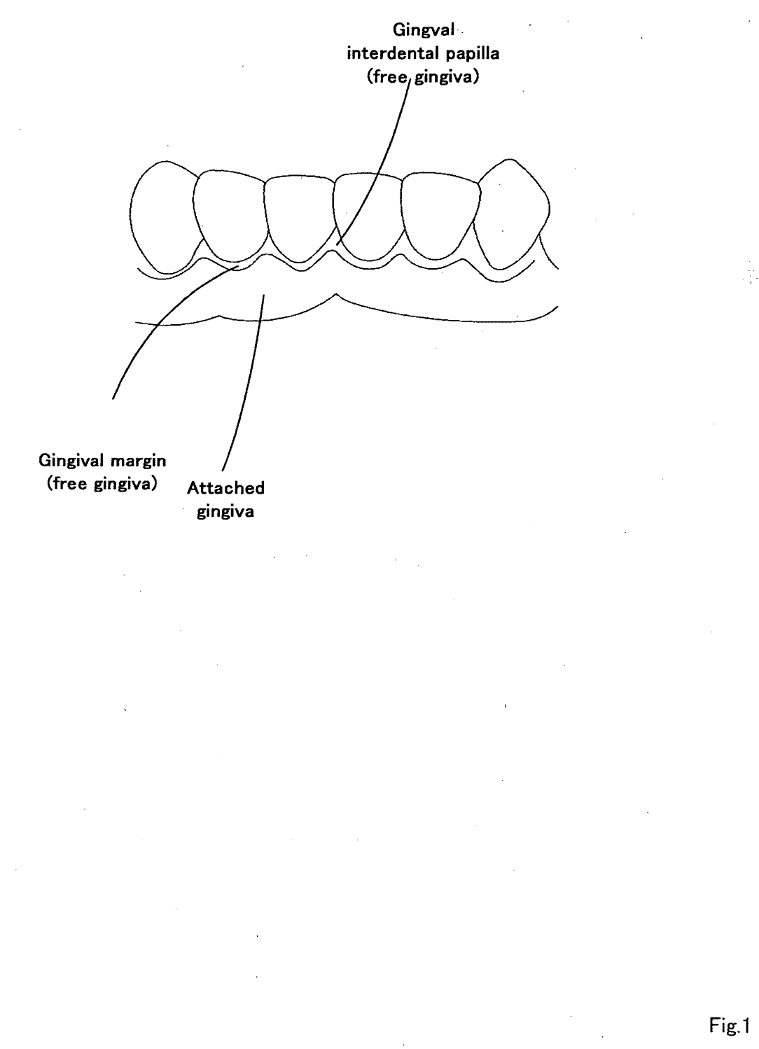

FIG. 2 is a schematic diagram of the measurement points for the site (elevated region) of subcutaneous injection of the composition of the present invention;

FIG. 3 is a graph that shows changes along time lapse (postoperative day 0, day 6, and day 12) in the height of the injection site formed when a test liquid was injected into the dorsal region of a nude mouse;

FIG. 4 is a graph that shows changes along time lapse (postoperative day 0, day 6, and day 12) in the volume of the injection site formed when the test liquid was injected into the dorsal region of the nude mouse;

FIG. 5 is a graph that shows changes along time lapse (postoperative day 0, day 6, and day 12) in the major axis length of the injection site formed when the test liquid was injected into the dorsal region of the nude mouse;

FIG. 6 is a graph that shows changes along time lapse (postoperative day 0, day 6, and day 12) in the minor axis length of the injection site formed when a test liquid was injected into the dorsal region of the nude mouse;

FIG. 7 is a figure that shows the course of gingival interdental papillary regeneration in a male patient in Example 4: picture a shows the preoperative interdental papilla; picture b shows the interdental papilla immediately after injection; picture c shows the interdental papilla 1 week after injection; and picture d shows the interdental papilla 3 months after injection; and

FIG. 8 shows micrographs of the tissue at the elevated region in the mice in Example 3: picture a is a micrograph image (40×) after HE staining; picture b is a micrograph image (400×) after HE staining; and picture c is a micrograph image after fluorescent staining in which the orange color indicates implanted MSCs, the blue color indicates nuclei, and the green color indicates collagen that has been produced.

### BEST MODE FOR CARRYING OUT THE INVENTION

The periodontal soft tissue regenerating composition of the present invention characteristically contains cells selected from mesenchymal stem cells and gingival fibroblasts, and a matrix material. The periodontal soft tissue regenerating composition of the present invention makes possible the low-invasive regeneration of periodontal soft tissue by avoiding the burden on the patient associated with the acquisition and implantation of graftable tissue. In addition, the composition of the present invention makes it possible to regenerate the free gingiva, which has heretofore been difficult, and can readily regenerate highly aesthetic gums.

The composition of the present invention is also useful for the regeneration of the interdental papilla. The interdental papilla is susceptible to inflammation and is prone to produce a gap through, for example, gingival recession. The interdental space, however, has been refractory to surgical procedures because it presents a narrow space for the execution of a free graft or a pedicle graft, and also because the blood supply to the graft is not adequate and there are limitations due to the surrounding dental roots and crowns. However, the present invention, through the injection of the present composition into periodontal tissue, can readily bring about regeneration of the interdental papilla without carrying out transplantation of graft of an alveolar bone or gingiva.

Embodiments of the composition of the present invention for regenerating periodontal soft tissue and embodiments of the method of producing this composition are described in the following.

The Periodontal Soft Tissue Regenerating Composition

The composition of the present invention for regenerating periodontal soft tissue (also referred to hereafter simply as the present ‘composition’) comprises cells selected from mesenchymal stem cells and gingival fibroblasts, and a matrix material. The present composition can be applied to humans and non-human mammals, and is preferably used with humans.

The Mesenchymal Stem Cells And Gingival Fibroblasts

The present composition can contain mesenchymal stem cell(s). Mesenchymal stem cell is a somatic stem cell and is a cell that has a self-replication capacity and a multipotent capacity to differentiate into a number of mesenchymal cells. Mesenchymal stem cell is obtained according to standard methods by harvesting from, for example, bone marrow, periosteum, peripheral blood, umbilical cord blood, adipose cells, and so forth, followed by culturing and work up. For bone marrow, refer to Mark F. Pittenger et al., Science (1999) Vol. 284, pp. 143-147 and Katia Mareschei et al., Haematologica (2001) Vol. 6, pp. 1099-1100; for peripheral blood, refer to Sergei A. Kuznetsov et al., The Journal of Cell Biology (2001) Vol. 153, pp. 1133-1139; for umbilical cord blood, refer to Alejandro Erices et al., British Journal of Haematology (2000) Vol. 109, pp. 235-242; and for adipose cells refer to Patricia A. Zuk et al., Tissue Engineering (2001) Vol. 7(2), pp. 211-228. In the case of the present invention, harvesting from oral cavity tissue is also preferred given that the goal is the regeneration of periodontal tissue or considering the convenience of harvesting. This oral cavity tissue can be exemplified by alveolar bone marrow, the palate, alveolar bone periosteum, dental follicular tissue, periodontal ligament, alveolar bone marrow, and so forth. These cells can also be recovered from dental pulp cells or dental follicular cells derived from, e.g. the gingiva acquired accompanying, e.g. tooth extraction.

The present composition may also contain gingival fibroblasts. The gingival fibroblasts may be cells that have been induced to differentiate so as to express a gingiva forming capacity, such as gingival fibroblasts from undifferentiated/stem cells such as mesenchymal stem cells. For example, differentiation can be induced by basic fibroblast growth factor (bFGF).

One or more selected from these mesenchymal stem cells and gingival fibroblasts can be used in the present invention. Thus, only mesenchymal stem cells may be used, or only gingival fibroblasts may be used, or both of them may be used. In addition, these cells may be cultured cells. In relation to the individual in whom the present composition is used, the cells used by the present invention encompass autologous cells, allogeneic cells and xenogeneic cells; whereas allogeneic cells are preferred, and autologous cells are more preferred. For example, when the goal is the regeneration of human periodontal soft tissue, the use is preferred of autologous cells, or allogeneic cells that exhibit a large number of matching histocompatibility antigens.

The Matrix Material

The matrix material used in the present composition is a matrix material that has the capacity to form, prior to its application (administration) or in vivo, a gel or fluid that exhibits a suitable viscosity. A matrix material that undergoes degradation and absorption in vivo is preferred. The matrix material can be, for example, a polymeric material heretofore used as a scaffold for cells. It can be exemplified by synthetic polymers such as polylactic acid, polyglycolic acid, lactic acid/glycolic acid copolymers, poly-ε-caprolactone, ε-caprolactone/lactic acid or glycolic acid copolymers, polycitric acid, polymalic acid, poly-α-cyanoacrylate, poly-β-hydroxybutyric acid, polytrimethylene oxalate, polytetramethylene oxalate, polyorthoester, polyorthocarbonate, polyethylene carbonate, polypropylene carbonate, poly-γ-benzyl-L-glutamate, poly-γ-methyl-L-glutamate, poly-L-alanine, and so forth; polysaccharides such as starch, alginic acid, hyaluronic acid, chitin, pectinic acid, and derivatives of each of the preceding; and peptides and proteins such as gelatin, collagen (any type of collagen to which any extraction procedure may be used), albumin, fibrin, fibrinogen, fibronectin, vitronectin, and so forth. For each of the preceding matrix materials, a crosslinked product therefrom or a derivative yielded by partial chemical modification may be used. Various commercially available gels may also be used (Matrigel (brand name), BD PuraMatrix Peptide Hydrogel, both from Becton, Dickinson and Company). Such matrix material can function as a retentive carrier for the cells, growth factors, and so forth at the site where the composition of the present invention is injected. These matrix materials are denoted as non-blood-coagulation matrix materials in contrast to the blood-coagulation-system matrix materials described below.

According to knowledge held by the present inventors, these non-blood-coagulation matrix materials exhibit an excellent capacity in penetrating to the injection site and an excellent maintenance (retention) at the injection site and can readily cause penetration by the present composition into the tissue at the injection site. This makes it possible to have the present composition arrive at the site requiring regeneration (or the neighborhood thereof) with a preferred injection site configuration, e.g., having an efficiently and smoothly elevated surface. When it is difficult for the present composition to penetrate into the tissue, an elevated region may be formed while being accompanied by a plurality of small bumps on the surface at the injection site, whereby the injection site ends up being formed in a manner unsuitable for tissue regeneration; moreover, in such a case, it becomes quite difficult for the present composition to reach the defect sites to be regenerated and bring about the corresponding tissue regeneration.

Also usable as the matrix material are blood-coagulation-system matrix materials derived from a blood fraction that contains plasma and/or platelets. Plasma contains adhesion factors, e.g., fibrin, fibrinogen, fibronectin, and so forth, that are suitable as the scaffolding for the fibroblasts and mesenchymal cells of soft tissue. Platelets contain factors that promote fibrinogen formation and enable the formation of a viscous fluid or gel by releasing these factors when subjected to certain stimuli in vivo or in vitro.

According to knowledge held by the present inventors, it is believed that the blood-coagulation-system matrix materials are effective for tissue regeneration, despite that they have poor penetration behavior and poor retention behavior.

The platelets may be solely used by themselves, and the plasma may as well be solely used by itself. However, the platelets are preferably used in combination with fibrin and/or fibrinogen, and are also preferably used in combination with plasma, which is another blood fraction. Platelet-rich plasma (PRP), in which the platelets have been concentrated, is preferably used as a blood fraction that contains plasma and platelets. Platelet-rich plasma contains adhesion factors of plasma origin, e.g., fibrin, fibrinogen, and so forth, and its viscosity is readily increased by the addition of, for example, calcium chloride/thrombin, which causes gelation and thereby enabling the formation of a carrier that retains platelets and cells in vivo.

PRP can be prepared, for example, in accordance with the method of Whitman et al. (Dean H. Whitman et al., J Oral Maxillofac Surg, 55, 1294-1299 (1997)) by subjecting collected blood to a centrifugal separation process. PRP is known to be rich in growth factors such as platelet-derived growth factor (PDGF), transforming growth factor β1 (TGF-β1), transforming growth factor β2 (TGF-β2), and so forth (Jarry J. Peterson, Oral Surg Oral Med Oral Pathol Oral Radiol Endod, 85, 638-646 (1998)).

An example of the method of preparing PRP is as follows. An anticoagulant such as sodium citrate is first added to collected blood, which is then allowed to stand for a prescribed period of time at room temperature. After this, a centrifugal treatment is carried out under conditions (for example, about 5,4000 rpm) that separate the erythrocytes and buffy coat. This results in separation into two layers (in which the upper layer is called the platelet-poor plasma and the lower layer contains erythrocytes and the buffy coat). After removal of the upper layer, a centrifugal treatment is carried out under conditions (for example, about 2,400 rpm) that separate the erythrocytes. As a result, a fraction (platelet-rich plasma or PRP) that substantially does not contain the separated erythrocytes is recovered. The method of producing PRP is not limited to the aforementioned method, and the production can be carried out by a method that has been modified as necessary. Moreover, PRP from autologous blood is preferably used. This eliminates the risk of toxicity or an immune rejection reaction.

There is no general definition of the platelet count in PRP (i.e. platelet concentration rate), but plasma containing about 150-times to about 1500-times that in the collected blood may be used as the PRP in the present invention. The platelet concentration rate of the PRP in the present invention is given by the following formula:

platelet concentration rate(%)=(average platelet count in the PRP/average platelet count in the starting whole blood)×100

Accordingly, when, for example, the average platelet count in the PRP is 1,000,000 and the average platelet count in the whole blood is 300,000, the platelet concentration rate is approximately 333%. The platelet concentration rate for the PRP is preferably in the range of about 150% to about 1,500% (when converted into the average platelet count, this generally corresponds to about 240,000/μL to about 6,150,000/μL). A more preferred platelet concentration rate is in the range of about 300% to about 600% (when converted into the average platelet count, this generally corresponds to about 480,000 μL to about 2,460,000 μL).

The platelet concentration rate for the PRP can be adjusted by suitable adjustment of the centrifugal treatment conditions during PRP production. For example, when a two-stage centrifugal treatment as described above is carried out, a PRP for which the platelet concentration rate is in the range of about 300% to about 600% can be obtained by executing the first centrifugal treatment at about 500 rpm to about 1500 rpm (for example, 1,100 rpm) for about 5 minutes to about 15 minutes (for example, about 5 minutes) and executing the second centrifugal treatment stage at about 2000 rpm to about 5000 rpm (for example, about 2,500 rpm) for about 5 minutes to about 15 minutes (for example, about 5 minutes). The platelet concentration rate for the PRP can be measured by the usual methods (for example, using the commercially available Sysmex XE-2100 (Sysmex, Tokyo, Japan) as shown in the examples).

PRP is also preferred because it is an autologous material that can be produced from peripheral blood that has been collected from the individual in whom the present composition is to be used. In addition, as described below, PRP is also preferred for its rich content of cell growth factors. The PRP can be produced, for example, from collected peripheral blood utilizing the usual methods, however, modifications may be introduced as necessary by the individual skilled in the art. When the PRP is produced as an autologous material, autologous thrombin and/or autologous serum collected from the peripheral blood of the concerned individual is preferably added. The PRP can be used in the form of a 1:1 to 10:1 mixture with CaCl₂/thrombin. A composition that can induce the blood coagulation system (e.g., platelets, mixtures of platelets and fibrin and/or fibrinogen, mixtures of platelets and plasma (in a form other than platelet-rich plasma)) can also be used alternately as a PRP equivalent in place of PRP.

The matrix material can be a single matrix material as described above or can be a combination of two or more of the matrix materials as described above. According to knowledge held by the present inventors, a non-blood-coagulation system matrix material is preferably used as the matrix material in order to effectively obtain excellent tissue penetrance and retention at the injection site for periodontal tissue. More preferably, a selection is made from (a) hyaluronic acid and derivatives thereof, (b) collagen and derivatives thereof, and (c) fibrin and fibrinogen. The matrix material is even more preferably selected from (a) hyaluronic acid and derivatives thereof and (b) collagen and derivatives thereof. On the other hand, the matrix material may preferably be selected from blood-coagulation-system matrix materials, such as (d) platelets and plasma and preferably PRP, in order to effectively obtain cell growth and a tissue regeneration capacity in periodontal tissue.

In order to obtain a highly aesthetic gingiva, the matrix material is preferably selected from the (a) hyaluronic acid and derivatives thereof. The use of hyaluronic acid and so forth can prevent discoloration due to the matrix material itself and can bring about a rapid regeneration of gingival tissue that exhibits the original gingival color. The use of mesenchymal stem cells is preferred in this case. When a combination of hyaluronic acid and mesenchymal stem cells is used, it is preferably used in combination with a blood coagulation-type matrix material, e.g., PRP, as described below.

Viewed from the perspective of regenerating the free gingiva, the matrix material is preferably selected in particular from the (b) collagen and derivatives thereof. The use of gingival fibroblasts is preferred in this case. As is shown in the examples described below, a composition comprising gingival fibroblasts and the (b) collagen and derivatives thereof as the matrix material exhibits a high amount of gingival regeneration even in the absence of the blood coagulation-type matrix material of the (d). This is due to a synergistic effect from the combination of the collagen with the gingival fibroblasts, and is an effect that is not obtained with either component by itself. In addition, this composition can easily fill even deficits such as in the interdental gingiva, for example, the black triangle.

Again viewed from the perspective of regenerating free gingival tissue, the matrix material is preferably one or more selected from the (a) hyaluronic acid and derivatives thereof and (b) collagen and derivatives thereof and one or more selected from (d) platelets and plasma such as PRP. In this case, the cells can be mesenchymal stem cells and/or gingival fibroblasts; however, mesenchymal stem cells are preferred when the matrix material is selected from (a) hyaluronic acid and derivatives thereof while gingival fibroblasts are preferred when the matrix material is selected from (b) collagen and derivatives thereof. In the case under consideration, the matrix material can be selected from both (a) hyaluronic acid and derivatives thereof and (b) collagen and derivatives thereof or can be selected from only one of (a) and (b). Viewed from the perspectives of the aesthetics and regenerating the free gingiva, the use is preferred of matrix material selected from (a) hyaluronic acid and derivatives thereof in combination with matrix material selected from (d) platelets and plasma such as PRP.

The blood coagulation matrix material can provide gingival regeneration merely by being combined with mesenchymal stem cells and/or gingival fibroblasts; however, it is preferably used in combination with the non-blood-coagulation system matrix material. By doing this, the cells penetrate and reach to and be retained at the intended site with blood coagulation matrix material and then start to accomplishes its functions.

Accordingly, the present composition preferably contains as its matrix material PRP and/or a PRP equivalent (referred to below simply as “PRP and the like”) as the blood-coagulation-system matrix material of (d) together with another matrix material (non-blood-coagulation system matrix material). The non-blood-coagulation system matrix material is preferably selected from (a) hyaluronic acid and derivatives thereof and (b) collagen and its derivatives. A viscosity suitable for injection can be obtained by producing a composition comprising the cells+blood-coagulation-system matrix material (PRP and the like)+non-blood-coagulation system matrix material and obtained by mixing the preceding to homogeneity.

Because such a composition has a composite matrix material of blood-coagulation-system matrix material (PRP and the like) mixed with non-blood-coagulation system matrix material, the present composition can readily penetrate into the tissue at the injection site. This makes it possible to have the present composition arrive at the site requiring regeneration (or the neighborhood thereof) with a preferred injection site configuration, e.g., having an efficiently and smoothly elevated surface. In addition, the presence of the composite matrix comprising PRP and the like and other matrix material results in excellent retention by the PRP and the like as well as the cells at the injection site. As a result, an excellent cell growth behavior and an excellent tissue regeneration performance can be secured at the injection site for the present composition and an effective regeneration can be brought about not only for the attached gingiva but also for the free gingiva. Thus, the use of a composite matrix comprising PRP and the like enables even deficits such as in the interdental gingiva, for example, the black triangle, to be easily filled.

Based on the preceding teachings, when the present composition comprises a blood coagulation matrix material (PRP and the like) and a non-blood-coagulation matrix material, the composition preferably contains the non-blood-coagulation matrix material and the blood coagulation matrix material each at a degree that secures penetrance and retention when the present composition is injected into tissue. When there is too little non-blood-coagulation system matrix material or too much blood coagulation matrix material, penetration into the tissue at the time of injection is impaired, resulting in a tendency for the formation of tissue and an injection site shape that have fine peaks and valleys, and retention of the cells and PRP and the like at the injection site is impaired, resulting in a tendency for the tissue regeneration performance at the injection site to decline.

A preferred mixing ratio between the blood coagulation matrix material (PRP and the like) and the non-blood-coagulation matrix material in the present composition can be established, for example, based on the capacity to retain an elevated shape at the subcutaneous injection site as indicated by the height or volume of the injection site formed when the present composition is injected (described below). For example, PRP having a platelet content in the normal range is preferably used in a ratio of from 10 v/v % (inclusive) to 900 v/v % (inclusive) with reference to a 1 to 3% collagen solution (preferably a 2% collagen solution) or a 3 mg/mL to 15 mg/mL hyaluronic acid solution (preferably 10 mg/mL). At least 100 v/v % is more preferred and at least 200 v/v % is even more preferred. The effect from the co-use of PRP and other matrix material can be reliably obtained when at least 100 v/v % is employed, while the use of at least 200 v/v % facilitates the generation of a fine tissue condition when the present composition is injected. In addition, no more than 250 v/v % is preferred. This is because the appearance of the co-use effect is spoiled at above 250 v/v %. In those instances where the combination with PRP is employed, the collagen concentration in the vehicle or composition is preferably at least 0.2% but no more than 1.8% and more preferably is no more than 1% and even more preferably is no more than 0.6%. The hyaluronic acid concentration in the vehicle or composition is preferably at least 1 mg/mL but no more than 9 mg/mL, and more preferably is no more than 5 mg/mL, and even more preferably is no more than 3 mg/mL.

Examples of preferred matrix compositions for the present composition are shown in Table 1. These compositions are preferred for periodontal soft tissue, but in particular are compositions preferred for the regeneration of the free gingiva and particularly for the gingival interdental papilla. The free gingiva can be effectively regenerated by the combination of mesenchymal stem cells+hyaluronic acid+PRP and the like and by the combination of fibroblasts and collagen. Each of the compositions shown in Table 1 may contain both mesenchymal stem cells and fibroblasts.

TABLE 1

| | Matrix Material | |
|---|---|---|
| | non-blood-coagulation system | blood-coagulation-system |
| kind of cell | matrix material | matrix material |
| mesenchymal | hyaluronic acid and its derivatives, | Employed: platelets, PRP, PRP |
| stem cells | collagen and its derivatives, | and the like, preferably PRP |
| | preferably hyaluronic acid | |
| gingival | hyaluronic acid and its derivatives, | None |
| fibroblasts | collagen and its derivatives, | Emplyed: platelets, PRP, PRP |
| | preferably hyaluronic acid | and the like, preferably PRP |

Cell Growth Factors

The present composition can contain a cell growth factor. This cell growth factor is a cell growth factor that promotes gingival regeneration by the cells present in the present composition, but is not otherwise particularly limited. It can be exemplified by cell growth factors that promote cell growth and wound healing. Furthermore, it may preferably be a cell growth factor that is present e.g. in the α-granules of platelets. Such growth factors can be exemplified by PDGF (promotes cell growth), TGF-β (promotes the production of type IV collagen, which stimulates the cell cycle), VEGF, EGF, bFGF, and the like.

The present composition may contain such growth factors in the form of platelets or in the form of plasma plus platelets and preferably in the form of PRP. As already noted, PRP can be employed as a matrix material in the present composition, but it can also be utilized as a source of cell growth factors.

Because the composition of the present invention contains matrix material in combination with cells that have the gingiva-forming capacity, it readily reaches the site requiring regeneration and provides excellent retention of the cells at this site. The periodontal soft tissue can be readily regenerated as a result. When the composition of the present invention is employed to regenerate free gingiva as the particular periodontal soft tissue, it preferably has a capacity to maintain an elevated shape at the injection site. This is because the free gingiva itself assumes a freestanding or elevated configuration. An index of the capacity to maintain an elevated shape at the injection site can be, for example, the height or volume of the injection site that is formed when the composition of the present invention is injected subcutaneously in the dorsal region of a rodent, e.g. the mouse or rat, and preferably into a rodent used as a laboratory animal.

An example of such an index is the ratio of the height of the injection site after a prescribed time period to the height of the injection site at the time of injection. This is because the height is a suitable index of the capacity of the grafted tissue to provide elevation or a freestanding feature. This height ratio can be determined, for example, as follows:

(a) The ratio of the height of the elevated region at 144 hours after injection of the composition, with respect to the height at the time of injection of the elevated region formed when the composition is subcutaneously injected into the dorsal region of a rodent.

In this measurement method, the rodent is preferably a laboratory animal and more preferably is a nude mouse. The amount of injection of the composition of the present invention can be 0.5 mL to 3.0 mL and is preferably 1.0 mL. The height of the elevated region is the maximum height of the elevated region where the composition has been subcutaneously injected in the dorsal region, and it is measured above markings using calipers.

The height ratio determined in this manner is preferably at least 15%. An elevated condition is readily maintained even afterwards when this height ratio is at least 15%. At least 30% is more preferred.

Another aspect of the height ratio is the ratio of the height of the elevated region at 288 hours after injection of the composition of the present invention with respect to the height of the elevated region at the time of injection. The measurement method is the same as above. This height ratio is preferably at least 15%. The elevated condition is even more readily maintained when this height ratio is at least 15%. At least 30% is more preferred. For the composition of the present invention of this aspect, the ratio of the height of the elevated region at 288 hours after injection of the composition to the height of the elevated region at the time of injection is even more preferably at least 10%.

An example of another index is the ratio of the volume of the injection site after a prescribed time period with respect to the volume of the subcutaneous injection site at the time of injection. This is because the volume is a suitable index of the capacity of the grafted tissue to provide elevation or a freestanding feature. This volume ratio can be determined, for example, as follows:

(b) The ratio of the volume of the elevated region at 144 hours after injection of the composition with respect to the volume at the time of injection of the elevated region formed when the composition is subcutaneously injected into the dorsal region of a rodent.

The method of measuring this volume can also employ the same procedure as for measurement of the height, supra. For the volume measurement, markings are made at 4 or more locations on the outline of the area that is elevated when the composition of the present invention is injected, and based on these markings the long diameter and the short diameter of the elevated region are measured and its height is also measured. Assuming that the elevated region has a hemispherical or a semi-elliptical shape, an approximate value for the volume of the elevated region is calculated based on the data for the corresponding diameter or long diameter and short diameter. The markings are preferably disposed at locations where the long diameter and the short diameter and the outline of the elevated region intersect. The long diameter and the short diameter can be measured using a suitable instrument, for example, calipers.

The thusly determined volume ratio is preferably at least 10%, and this ratio is more preferably at least 20%. It is even more preferably at least 30%.

The present composition is preferably capable of being injected at the time of use at the periodontal tissue location, that is, by local subcutaneously injection. Particularly the noncellular components of the present composition may be soluble powders at the time of use and a preliminary preparation may be carried out so as to yield a fluidity suitable for injection, e.g., a liquid or gel, or the present composition may be a fluid prepared by a preliminary dilution at the time of use. In addition, it may be a kit comprising two or more agents that are deployed by mixing at the time of use. The present composition can contain a suitable solvent that is used at the time of injection. Such solvents can be exemplified by a physiologically compatible buffer solution, physiological saline, and various injectable solvents. The noncellular components of the present composition may as necessary include a stabilizer, preservative, pH modifier, thickener, and so forth.

Method of Producing the Present Composition

The method of preparing the present composition is not particularly limited and, for example, may just involve simply mixing the cells and matrix materials that are the constituent components of the present composition along with optional components such as cell growth factors and so forth. As necessary, a buffer solution, physiological saline, and/or an injectable solvent may be included. The cell count in the present composition is preferably about 1.0×10⁴ cells/mL to 1.0×10¹⁰ cells/mL.

The various matrix materials that have already been described can be used as a cell-buffering matrix, and preferably one or more selected from (a) hyaluronic acid and derivatives thereof, (b) collagen and derivatives thereof, (c) fibrin and fibrinogen, and (d) plasma and platelets (typically PRP) are used. The matrix material solution used for the present composition can have, for example, a hyaluronic acid concentration of 3 mg/mL to 15 mg/mL and preferably 10 mg/mL and can have a collagen concentration of 0.5 to 5 weight % and preferably 1 to 3 weight %.

With regard to the preparation of the present invention, the cells used must be preliminarily collected and prepared in the required amount. The collected cells may be cultured and expanded according to the usual methods in correspondence to the type of cell collected. Thus, cells selected from undifferentiated mesenchymal stem cells and gingival fibroblasts may be cultured and cells selected from the cultured undifferentiated mesenchymal stem cells and gingival fibroblasts may be mixed with the matrix material.

Cell Collection, Separation, and Culture

Cell collection, separation, culture, and so forth may be carried out by the usual methods, for example, as described below in the case of mesenchymal stem cells.

(1) Procedure for Collecting and Separating Bone Marrow-Derived Mesenchymal Stem Cells

Human or laboratory animal bone marrow can be collected by the usual methods from, for example, the ilium. In the case of collection from oral cavity bone marrow, a marrow fluid may be collected by perforating the alveolar bone until a marrow fluid drains out. This collected marrow fluid is seeded to a tissue culture dish along with the appropriate medium (for example, 10% FBS or DMEM medium containing autologous serum), and only the cells adhering to the culture dish after several days are cultured while the suspended cells are washed off.

(2) Procedure for Collecting and Separating Oral Cavity Periosteal Cells

The alveolar mucosa of the palate or maxilla or mandible of a human or laboratory animal is released and the periosteum on the alveolar bone is exposed and periosteum of a suitable size is collected; the collected periosteum is finely minced and thereafter is incubated with collagenase at 37° C. The cells are then dispersed by, for example, pipetting, and the are concentrated by filtration or centrifugal separation. The number of obtained cells is measured and they are seeded to a tissue culture dish with an appropriate medium (for example, 10% FBS or DMEM medium containing autologous serum).

(3) Procedure for Collecting and Separating Dental Pulp Tissue- or Dental Follicle Tissue-Derived Stem Cells

In collection from dental pulp tissue or dental follicle tissue, the tooth germ is collected in accompany of tooth extraction; this tissue is visually dissected and then submitted to explant culture; and only the attached cells are cultured and the floating cells are washed off.

(4) Culture of Mesenchymal Stem Cells

The various mesenchymal stem cells obtained as described above may be cultured using any culture medium suitable for the culture of such cells. As necessary, culture may be carried out with the addition of cell growth factors such as bFGF and so forth (J Bone Mineral Res. 20 (2005), pp. 399-409; Biochem. Biophys. Res. Commun. 288 (2001), pp. 413-419). Culture can be carried out under any conditions suitable for mammalian culture, but is preferably generally carried out at 37° C. in the presence of 5% carbon dioxide. Serial stem cell culture may be carried out by suitable methods known in the field of stem cell culture.

(5) Procedures for the Separation, Collection, and Culture of Gingival Fibroblasts

In the case of gingival fibroblasts, for example, small gingival fragments collected from human or laboratory animal gingiva are treated with trypsin to disperse the cells. The supernatant is taken off to provide a cell suspension. The cell count is measured and dilution with culture broth is carried out to give a suitable cell count (for example, 1×10²/mL) or the cells are seeded to a Petri dish by explant culture, whereas the culture is carried out in an incubator at 37° C. under 5% CO₂. Passage of culture is carried out after a monolayer sheet is formed. The passage culturing of fibroblasts can be carried out by suitable methods known in the field of fibroblast culture.

The present composition is preferably used by local injection in periodontal tissue and more preferably is used by local injection in human periodontal tissue. The injection site can be in the vicinity of periodontal soft tissue recession or in the vicinity of a site of a periodontal soft tissue defect or deficiency, and can be in the vicinity of a site where the free gingiva is deficient after conventional regenerative surgery directly mainly to hard tissue. Specific examples are the vicinity of root surfaces that have been exposed by gingival recession and the vicinity of interdental papilla deficits, e.g., the black triangle.

50 to 5000 μL and more preferably 500 to 2000 μL of the present composition having a cell concentration of 1.0×10⁴ to 1.0×10¹⁰ cells/mL is administered per administration site. The administration site may be facial or lingual or both. Administration may be carried out by transcutaneous subcutaneous injection, or the injection site may be opened by incision, in which the present invention may be injected or filled, and suturing may then be done in this case. A matrix material that can function as distinctive scaffold at the site may also be separately filled at the incision site. Administration may be performed at two or more sites at the same time.

The composition of the present invention for local injection in the periodontium is well suited for the regeneration of very small regeneration sites such as the gingival margin and the gingival interdental papillae. When the present composition is administered by transcutaneous subcutaneous injection without accompanying incision, under the cases in which it is solely injected or being unaccompanied by artifacts from other surgery in the gingival surface, the appearance of the gingival surface will not be substantially disturbed and the aesthetics will be maintained even during treatment. When, in particular, hyaluronic acid is used as the matrix material, desirable aesthetics are maintained beginning with the initial injection with almost no influence on the appearance (e.g. color and so forth) of the gingiva. In addition, due to the excellent tissue penetrance and retention by the cells when the composition of the present invention contains matrix material, a satisfactory tissue regeneration effect can be obtained even without a large number of administrations. Low invasiveness is thus also obtained in this context. In the case of local injection, even a single-time administration can provide excellent regeneration of the attached gingiva and free gingiva and filling of the interdental papilla.

The present composition can be administered to the site of administration independently of the conventional GTR or enamel matrix-based methods and independently of tissue graft implantation or surgery to cover exposed root surfaces. It may also be administered at the same time as the execution of these conventional techniques or procedures as a complement thereto. The present composition may also be administered after conventional surgery after the alveolar bone or attached tissue has undergone a certain degree of repair.

The present composition is also effective for the low-invasive regeneration/repair of periodontal soft tissue, i.e. the attached gingiva and the gingival interdental papilla, e.g. free gingiva. The present composition can effectively regenerate the free gingiva, such as the gingival margin and gingival interdental papilla, where regeneration has heretofore been difficult or unsatisfactory. As a result the present composition can regenerate gums that present good aesthetics. The present composition can thus be used as a composition for regenerating the free gingiva and specifically as a composition for regenerating the gingival margin and as a composition for regenerating the gingival interdental papillae.

The Method of Regenerating Periodontal Soft Tissue

A method of regenerating periodontal soft tissue is an example of an embodiment of the present composition. Thus, through the administration of the present composition to a site of periodontal soft tissue recession or a site of periodontal soft tissue deficit in an individual who requires periodontal soft tissue regeneration, for example, a human, the attached gingiva and/or the free gingiva at such sites can be regenerated and gums with good aesthetic presentation can be functionally reconstructed. The regeneration method with the present composition draws a line from methods of directly administering the composition to the sites of recession or deficiency; in fact the present composition may be administered to residual tissue remaining around the recessed or lacking space (void), and the periodontal soft tissue is then regenerated therein in a manner that fills such a space. Thus, this is fundamentally different from supplying a composition into the void itself (e.g. the deficit site) in order to regenerate the site. In particular, the gingival interdental papilla can be effectively regenerated by using the composition of the present invention, and specifically therewithin collagen+fibroblasts or hyaluronic acid+mesenchymal stem cells+PRP. Such a regeneration method can be executed in combination with hard tissue regenerative surgery (for example, GTR, an enamel matrix procedure, cell grafting, tissue fragment grafting) focusing on the alveolar bone. For example, it may be carried out at the same time as these regenerative surgeries or after these surgeries. The composition of the present composition used in the regeneration method, the method of administration, and so forth, are in accordance with that already described above.

While the periodontal tissue regeneration method uses the present composition, the individual constituent components encompassed by the present composition need not be injected in composition form; they may be injected and so forth as individual reagents, either individually or in combinations of two or more. For example, a cell suspension or a fluid containing cells and growth factor may be injected after the preceding injection of the matrix material, or vice versa. The procedure for separately administering one or more of the constituent components of the present composition may be set up as appropriate for the circumstances.

The Method of Evaluating Periodontal Tissue Regenerating Compositions

The present invention also provides a method of evaluating periodontal tissue regenerating compositions. Thus, a composition suitable for the regeneration of the free gingiva can be obtained by carrying out a step of evaluating the free gingival regeneration capacity of different compositions using the previously described indices of free gingival or gingival interdental papillary regeneration. The composition of the present invention can be subjected to this evaluation. The evaluation index can be the previously described “height ratio” and/or “volume ratio” of the elevated region yielded by subcutaneous injection. The evaluation also uses the previously described numerical values for these indices. For example, a capacity to form free gingiva can be confirmed when the height ratio is equal to or greater than the above-cited numerical value, while a capacity to form free gingiva is ruled out when the height ratio does not satisfy the above-cited numerical value.

The present invention additionally provides a method of producing a free gingiva regeneration composition, wherein this method includes the evaluation step of the aforementioned evaluation method. Thus, once a particular composition has been confirmed by this evaluation method to have the capacity to form free gingival tissue, a free gingiva regeneration composition can be produced based on the components of this composition.

### EXAMPLES

The present invention is specifically described herebelow with examples, but the present invention is not limited by the following examples.

### Example 1

### Preparation of Human Mesenchymal Stem Cells

Collection was carried out preoperatively from the iliac bone of the patient by the usual method. The collected bone marrow aspirate was seeded to a tissue culture dish along with a suitable culture medium. The suspended cells were removed and the cells were cultured and expanded with exchange of the culture fluid on a constant-interval schedule.

### Example 2

### Preparation of Human Gingival Fibroblasts

Collection was carried out preoperatively from the gingiva of the patient. Seeding to a culture dish was carried out by explant culture and culture was carried in an incubator at 37° C. under 5% CO₂. Culture, expansion, and the production of cell clumps was carried out by general methods (the same methods as for undifferentiated mesenchymal stem cells).

### Example 3

### Cell Implantation

In this example, the cells and the matrix material of each system example were injected into mice and chronological changes in the size of the elevated region were measured in order to evaluate the possibility for use as a regenerating material for the attached gingiva and/or the free gingiva.

(1) Preparation of the Experimental Animals

Diethyl ether anesthesia was induced in nude mice and the anesthetized state was maintained by the intraabdominal cavity injection (limited to 0.6 mL of the 10-fold dilution) with a tuberculin inoculation syringe of pentobarbital sodium (preliminarily diluted 10-fold, brand name: Somnopentyl). After anesthesia, fur removal was carried out with a commercial depilatory cream.

(2) Production of the Fluid Regeneration Compositions

To prepare the fluid regeneration compositions that were implanted in the experimental animals, fluid compositions (a total of 1.0 mL) were prepared using the previously prepared human mesenchymal stem cells (MSCs) or human gingival fibroblasts (FBs). Each system example included a control in the form of a liquid of only the corresponding noncellular components (collagen, hyaluronic acid, PRP+hyaluronic acid, and PRP). PKH26 staining was done prior to injection.

TABLE 2

| Components | | Collagen | | | HA | | | HA + PRP | | | PRP | | |
|---|---|---|---|---|---|---|---|---|---|---|---|---|---|
| kinds of | MSC | — | 1 × 10⁷ | — | — | 1 × 10⁷ | — | — | 1 × 10⁷ | — | — | 1 × 10⁷ | — |
| cells | s (cells/ml) | | | | | | | | | | | | |
| | FB (cells/ml) | — | — | 1 × 10⁷ | — | — | 1 × 10⁷ | — | — | 1 × 10⁷ | — | — | 1 × 10⁷ |
| composition | Collagen (0.2%) | 100 | 100 | 100 | — | — | — | — | — | — | — | — | — |
| of matrix | (ml) | | | | | | | | | | | | |
| material (v/ | HA (10 mg/ml) | | — | — | 100 | 100 | 100 | 50 | 50 | 50 | — | — | — |
| v %) | (ml | | | | | | | | | | | | |
| | PRP (ml) | — | — | — | — | — | — | 50 | 50 | 50 | 100 | 100 | 100 |

| | | | | | | | | | | | | |
|---|---|---|---|---|---|---|---|---|---|---|---|---|
| [symbol] ml | 1.0 | 1.0 | 1.0 | 1.0 | 1.0 | 1.0 | 1.0 | 1.0 | 1.0 | 1.0 | 1.0 | 1.0 |

“—” means “the cell or matrix material was NOT empoyed”
PRP was emplyed with 10 wt % CaCl₂ + thrombin

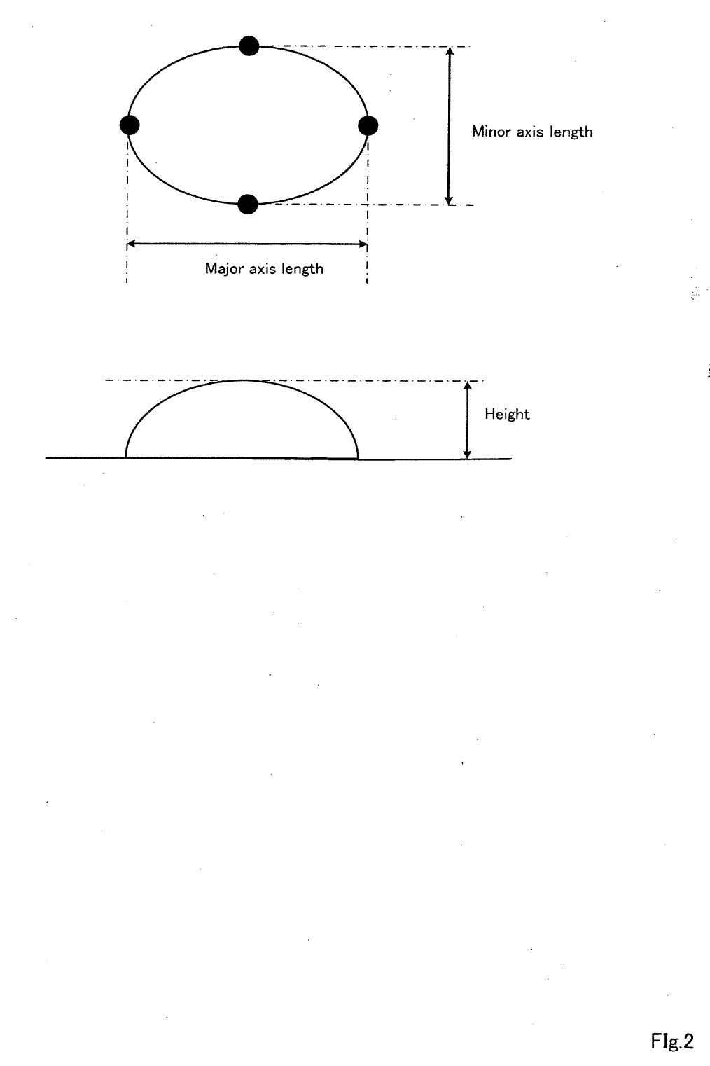

Each of the prepared liquids is shown in Table 2. Using a syringe with a 22-gauge needle, 1.0 mL of each of the three liquids was injected into the dorsal region of a single nude mouse. The circumference of the injection site was marked with black ink in order to clearly show the injection site. Thus, as shown in FIG. 2, the positions were marked on the edge of the injection site (protuberance) where the long diameter and the short diameter of the base of the protuberance were orthogonal to each other and the long diameter, short diameter, and height of the injection site were each measured and these values were designated as the measurement values on day 0 postoperative. Calipers were used to measure each of these dimensions.

(3) Postoperative Day 6

Each nude mouse was anesthetized in the same manner as at the time of injection and the size of the injection site was measured based on the location in the markings at the time of injection.

(4) Postoperative Day 12

Each nude mouse was sacrificed by excess diethyl ether inhalation. The size of the injection site was measured and the mouse dorsal region was removed. The extirpated fragments were examined under a microscope.

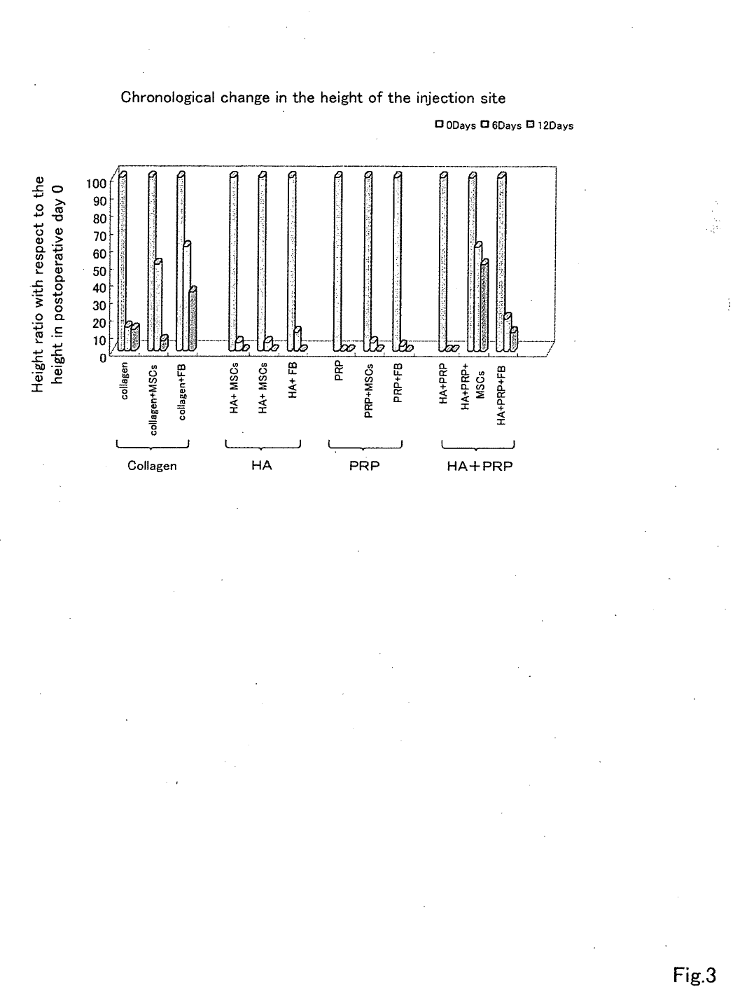

The injection site height, volume, major axial diameter, and minor axial diameter obtained from the measurement values on postoperative day 0, day 6, and day 12 are shown, respectively, in FIGS. 3 to 6. The elevated region was assumed to have a hemispherical or semi-elliptical shape and approximate values for the volume of the elevated region were calculated based on the data for the corresponding diameter, or the major axial diameter and minor axial diameter.

As shown in FIGS. 3 to 6, in all of the system examples the injection site, which was initially swollen following injection of the test liquid, underwent shrinkage with a reduction in its height, short diameter, and long diameter.

Analysis for the Height

As shown in FIG. 3, the collagen system example, which employed collagen as the matrix material, maintained an injection site height at day 6 postoperative (after 144 hours) to about the same degree (50 to 65%) with both the mesenchymal stem cell-containing liquid and the fibroblast-containing liquid; however, at day 12 postoperative (after 288 hours), shrinkage had occurred with the mesenchymal stem cell-containing liquid (10%) while the fibroblast-containing liquid maintained 40%. The conclusion may therefore be drawn that collagen+fibroblasts is a composition that provides a suitable height retention rate. In the case of the hyaluronic acid system, the height was already at or below 10% on postoperative day 6 for both the mesenchymal stem cells and the fibroblasts and the height of the injection site was thus almost completely extinguished. The same trends were seen with the PRP system example as for the hyaluronic acid system example. In contrast to this, the (hyaluronic acid+PRP) system example had a retention rate at postoperative day 6 (i.e. after 144 hours) of about 60% with the mesenchymal stem cell-containing liquid and about 20% with the fibroblast-containing liquid and had a retention rate at postoperative day 12 (i.e. after 288 hours) of about 50% with the mesenchymal stem cell-containing liquid and less than 10% with the fibroblast-containing liquid. Thus, it was demonstrated that the (hyaluronic acid+PRP) system example presented a height retention rate that was excellent both with mesenchymal stem cells and with fibroblasts, although the mesenchymal stem cells were shown to have the more preferred height retention rate.

Analysis for the Volume

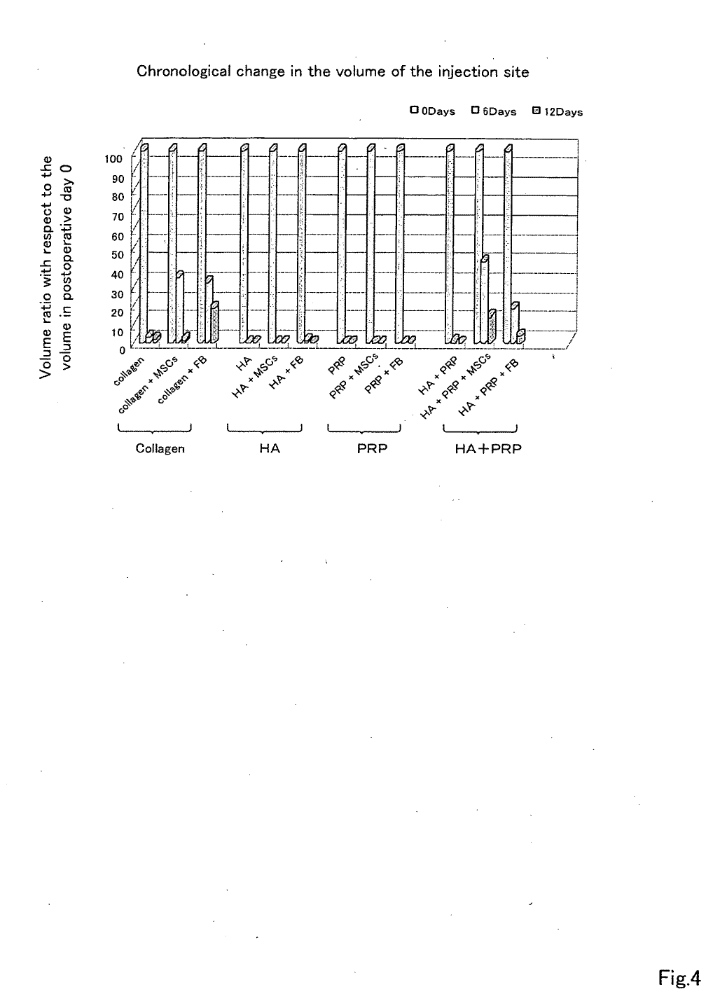

As shown in FIG. 4, the collagen system example, which employed collagen as the matrix material, maintained an injection site volume at postoperative day 6 (after 144 hours) to about the same degree with both the mesenchymal stem cell-containing liquid and the fibroblast-containing liquid; however, at postoperative day 12 (after 288 hours), the injection site had undergone almost complete retraction with the mesenchymal stem cell-containing liquid. In contrast to this, the fibroblast-containing liquid provided retention of about 30% of the volume with reference to the volume on postoperative day 0. That is, the conclusion may be drawn that collagen+fibroblasts is a composition that provides a suitable volume retention rate. In the case of the hyaluronic acid system, the volume of the injection site was already almost entirely lost at postoperative day 6 (after 144 hours) for both the mesenchymal stem cells and the fibroblasts. The same trend was seen with the PRP system example as for the hyaluronic acid system example. In contrast to this, the (hyaluronic acid+PRP) system example had a retention rate at postoperative day 6 (after 144 hours) of about 40% with the mesenchymal stem cell-containing liquid and about 16% with the fibroblast-containing liquid and had a retention rate at postoperative day 12 (after 288 hours) of more than 10% with the mesenchymal stem cell-containing liquid and less than 5% with the fibroblast-containing liquid. Thus, it was demonstrated that the (hyaluronic acid+PRP) system example presented a retention rate that was excellent both with mesenchymal stem cells and with fibroblasts, although the mesenchymal stem cells were shown to have the more preferred volume retention rate.

Analysis of the Long and Short Diameters

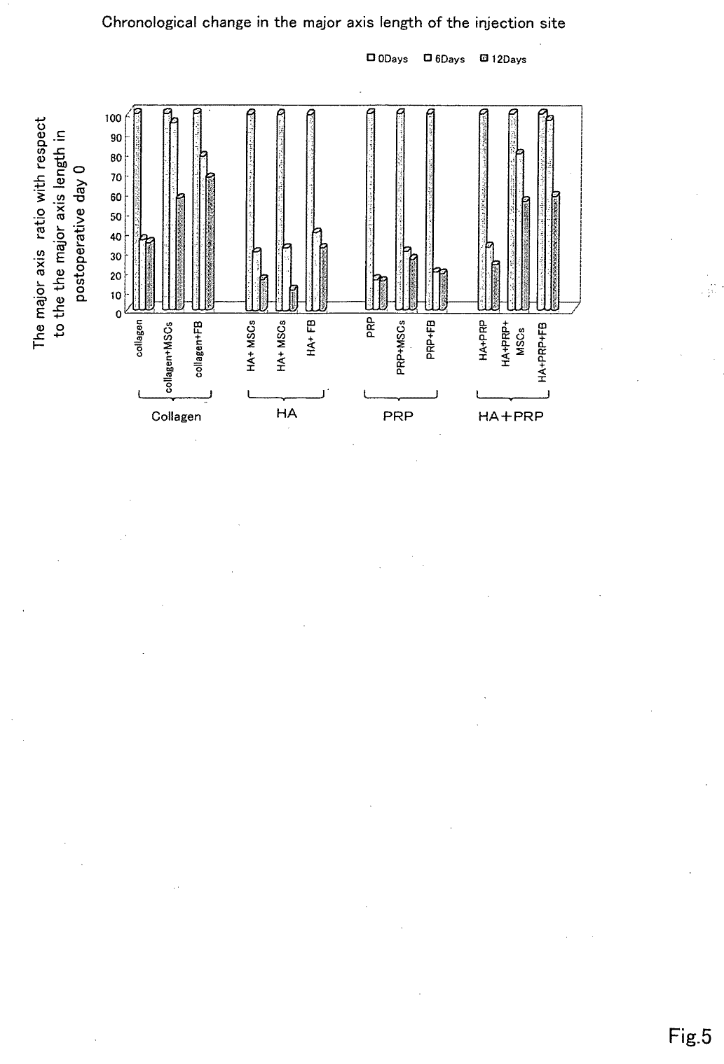

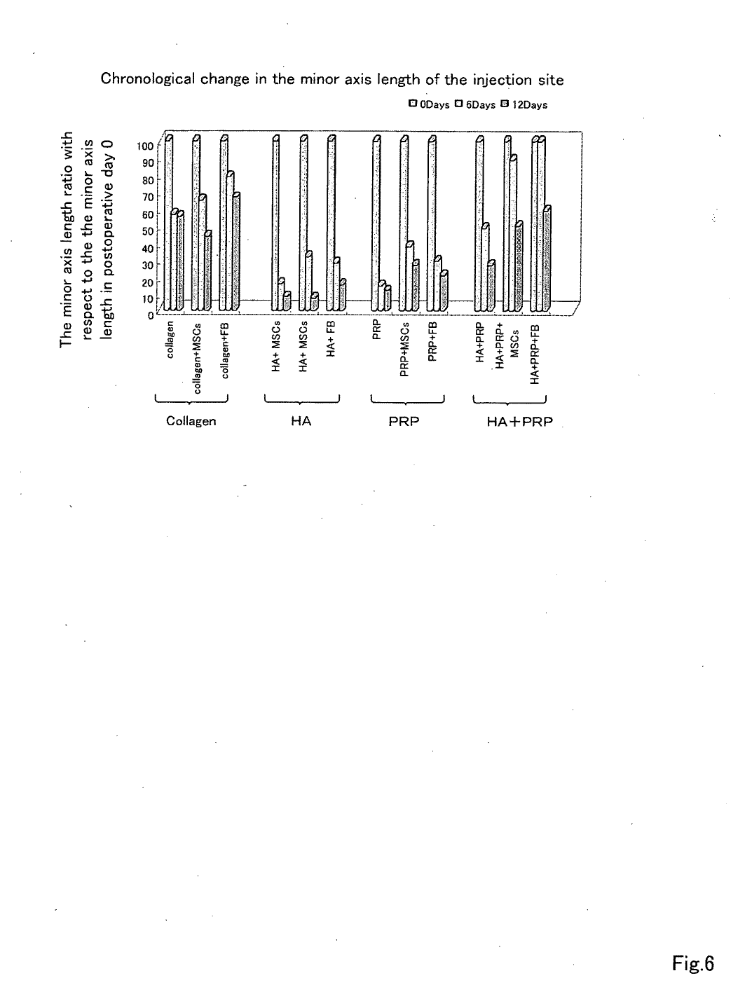

As shown in FIGS. 5 and 6, in all of the system examples the major axis length (long diameter) and the minor axis length (short diameter) also underwent reduction, however, all systems stayed at or above a certain value and differences within system examples and differences between system examples did not occur to the same degree as for the height and volume.

Based on the preceding results, it was demonstrated that a composition comprising at least a matrix material and mesenchymal stem cells or gingival fibroblasts could regenerate the attached gingiva among the periodontal soft tissues because the short diameter and long diameter of the elevated region stayed at or about a certain level with such a composition. In particular, it was demonstrated that the collagen+fibroblast combination and combinations containing hyaluronic acid and PRP (particularly the combination with mesenchymal stem cells) were clearly more effective than the other combinations with regard to the height and volume retention rates of the elevated region, and, in accordance with the profiles of their elevated regions, it was demonstrated that they are suitable for the regeneration not only of the attached gingiva, but also for the regeneration of the gingival interdental papillae of the freestanding gingival margin. In addition, it was demonstrated that the height and volume of the injection site correlate with each other and that, in addition to the volume of the injection site, the height of the injection side can be used as a convenience index of the free gingiva regeneration activity.

Based on the results of the observation of the subcutaneous injection site, it was demonstrated that the use of hyaluronic acid can provide a regenerated region with an excellent appearance in which the color of the mucosal surface presents almost no variation from that of the surroundings.

### Example 4

### Regeneration of the Gingival Interdental Papillae

This example is an example of the regeneration of the gingival interdental papillae by the injection of a test liquid containing mesenchymal stem cells into three patients presenting with black triangles due to a loss of gingival interdental papillae. The patients were as follows: male A (age: 54), who had a black triangle presentation due to an interdental papillary deficit in the mandibular dentition (specifically, the surrounding interdental papillae after a mandibular implant prosthesis); male B (age: 22), who had a black triangle presentation due to an interdental papillary deficit for a maxillary anterior tooth; and a 48-year old female who had a black triangle presentation due to an interdental papillary deficit in the maxillary dentition. MSCs collected from the bone marrow of each patient were cultured preoperatively. On the day of the procedure, 50 mL of blood was collected from each patient and the collected bloods were submitted to centrifugal separation to prepare PRP. The test liquids were prepared by respectively mixing 10 weight % CaCl₂+thrombin into a mixture prepared so as to have 100 v/v % to 230 v/v % PRP and 1×10⁷ cells/mL MSCs in a hyaluronic acid solution (10 mg/ml sodium hyaluronate). After preparation of the test liquids, a total of 1.0 mL was promptly injected in the vicinity of each of the above-cited deficit sites for each patent. In all the patients, regeneration of the gingival interdental papilla in the above-cited deficit sites had begun at 6 days post-injection and the black triangles were extinguished.

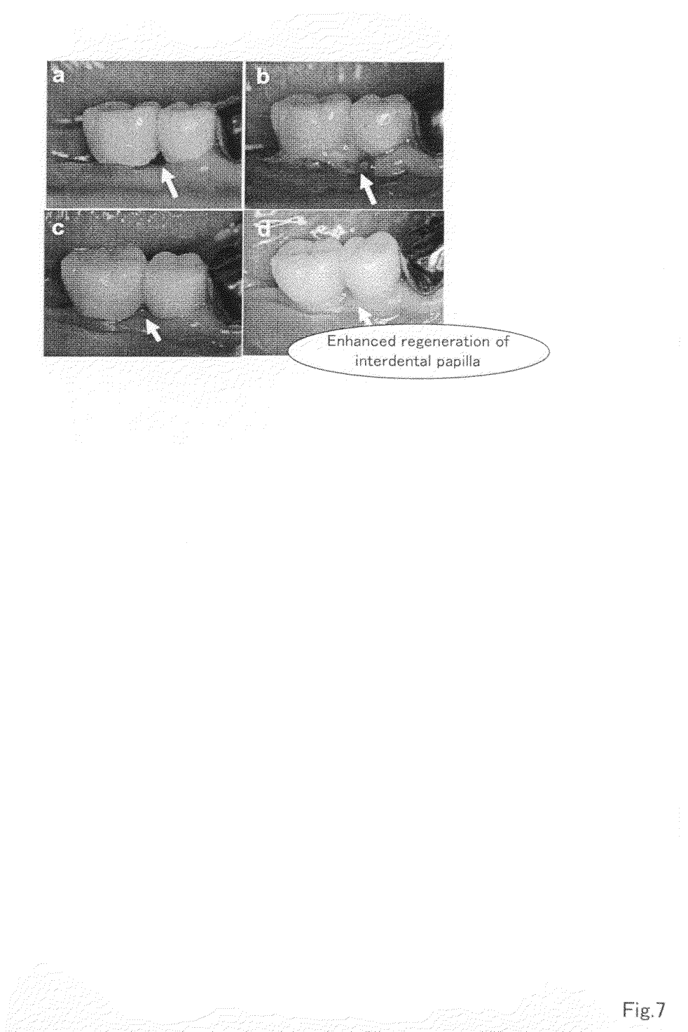

The results of observations preoperatively and with elapsed time postoperatively for the 54-year old male A are shown in FIG. 7. As shown in FIG. 7, while the black triangle presented preoperatively (refer to the picture a in FIG. 7), with each elapsed number of days postoperatively, gingival regeneration began, the interdental papilla was filled by regenerated gingiva, and the black triangle was extinguished.

### Example 5

### Subcutaneous Tissue Observations at the Injection Site in the Mice of Example 3

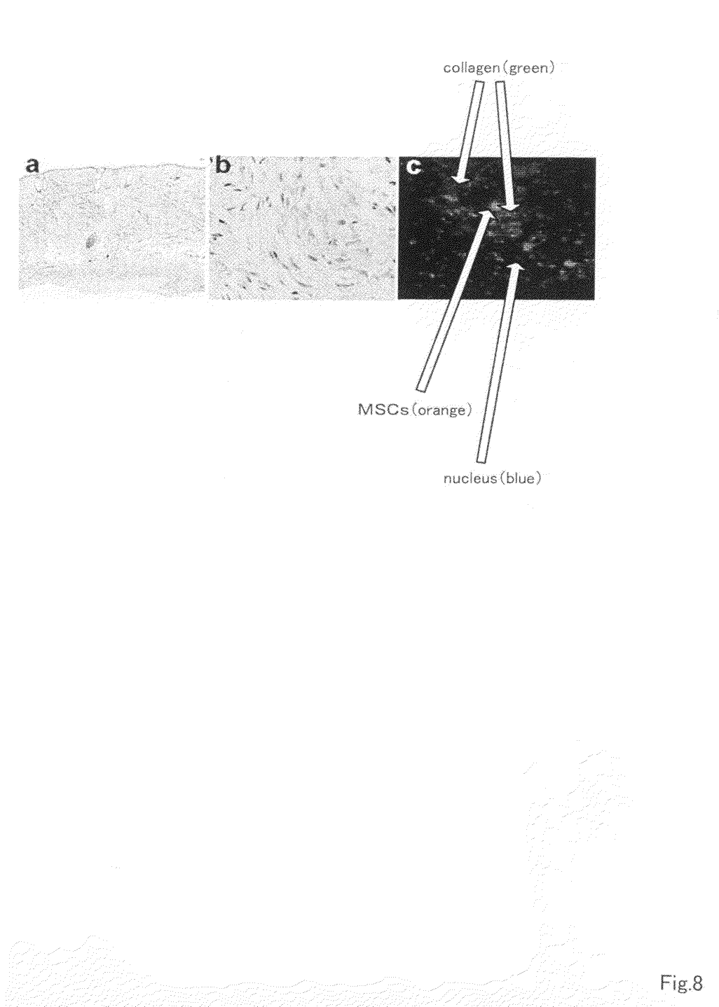

Tissue slices were collected from each injection site in the nude mice that were injected with the (MSCs/HA/PRP) composition prepared in Example 3 or the (FB/HA/PRP) composition, which are hyaluronic acid+PRP system examples. These specimens were submitted to HE staining and fluorescent staining, and the results of the staining are shown in FIGS. 8 and 9. a and b in FIG. 8 are micrograph images generated by HE staining, while FIG. 8c is a micrograph image generated by fluorescent staining. As shown in a to c of FIG. 8, for the (MSCs/HA/PRP) injection site the formation of good-quality tissue was demonstrated, as was a good production of type I collagen in the environment of the grafted MSCs (orange). In contrast to this, as shown in FIG. 9, there was less production of type I collagen in the tissue collected from the (FB/HA/PRP) injection site, which is also a hyaluronic acid+PRP system example, than for the (MSCs/HA/PRP). Based on these results, it was demonstrated that the (MSCs HA/PRP) composition provides an excellent amount of collagen production and that the results of the height ratio and volume ratio for the injection site (refer to Examples 3 and 4) correspond to the amount of collagen production.

The preceding results demonstrated that, through the use of a regenerative composition that has a good capacity to maintain an elevated shape, a convenient and highly aesthetic regeneration can be achieved even at difficult-to-regenerate locations such as the gingival interdental papillae.

This application bases its priority on Japanese Patent Application Number 2006-115932 filed 19 Apr. 2006, and the entire contents thereof are incorporated in this Specification.

1. A composition for regenerating periodontal soft tissue, comprising:
cells selected from mesenchymal stem cells and gingival fibroblasts; and
a matrix material.

2. The composition according to claim 1, further comprising a cell growth factor.

3. The composition according to claim 1, wherein the matrix material comprises one or more components selected from the following (a) to (d):
(a) hyaluronic acid and derivatives thereof
(b) collagen and derivatives thereof
(c) fibrin and fibrinogen
(d) plasma and platelets

4. The composition according to claim 3, wherein the matrix material is selected from at least (a) hyaluronic acid and derivatives thereof.

5. The composition according to claim 3, wherein the cells are mesenchymal stem cells.

6. The composition according to claim 3, wherein the matrix material is selected from at least (b) collagen and derivatives thereof.

7. The composition according to claim 3, wherein the cells are gingival fibroblasts.

8. The composition according to claim 3, wherein the matrix material is selected from at least (d) plasma and platelets.

9. The composition according to claim 3, wherein the matrix material is selected from each of (a) hyaluronic acid and derivatives thereof and (d) plasma and platelets.

10. The composition according to claim 9, wherein the cells are mesenchymal stem cells.

11. The composition according to claim 10, wherein the matrix material from (d) plasma and platelets is platelet-rich plasma.

12. The composition according to claim 11, wherein the matrix material is selected from each of (b) collagen and derivatives thereof and (d) plasma and platelets.

13. The composition according to claim 12, wherein the cells are gingival fibroblasts.

14. The composition according to claim 13, wherein the matrix material is selected from each of (b) collagen and derivatives thereof and (d) plasma and platelets.

15. The composition according to claim 1, wherein
when the composition is subcutaneously injected in a dorsal region of a rodent, a ratio for the height of an elevated region thus formed at 144 hours after injection to the height of the elevated region at the time of injection is at least 15%.

16. The composition according to claim 15, wherein the height ratio is at least 30%.

17. The composition according to claim 1, wherein
when the composition is subcutaneously injected in a dorsal region of a rodent, a ratio for the height of an elevated region thus formed at 288 hours after the injection of the composition to the height of the elevated region at the time of injection is at least 15%.

18. The composition according to claim 17, wherein the height ratio is at least 30%.

19. The composition according to claim 1, wherein, when the composition is subcutaneously injected in a dorsal region of a rodent, a ratio for the volume of an elevated region thus formed at 144 hours after the injection of the composition to the volume of the elevated region at the time of injection is at least 10%.

20. The composition according to claim 19, wherein the volume ratio is at least 20%.

21. The composition according to claim 1, wherein the periodontal soft tissue is attached gingiva.

22. The composition according to claim 1, wherein the periodontal soft tissue is free gingiva.

23. The composition according to claim 1, wherein the periodontal soft tissue is interdental papilla.

24. The composition according to claim 1, which is used for local injection in periodontal tissue.

25. A method of producing a composition for regenerating periodontal soft tissue, comprising steps of:
culturing cells selected from mesenchymal stem cells and gingival fibroblasts; and
mixing a matrix material with the cultured mesenchymal stem cells and/or gingival fibroblasts.

26. The production method according to claim 25, wherein the cells are mesenchymal stem cells or gingival fibroblasts.

27. The production method according to claim 25, wherein the periodontal soft tissue is attached gingiva.

28. The production method according to claim 25, wherein the periodontal soft tissue is free gingiva.

29. The production method according to claim 25, wherein the mixing step uses, as the matrix material, one or more components selected from the following (a) to (d):
(a) hyaluronic acid and derivatives thereof
(b) collagen and derivatives thereof
(c) fibrin and fibrinogen
(d) plasma and platelets

30. The production method according to claim 29, wherein the mixing step uses one or more components selected from at least (a) hyaluronic acid and derivatives thereof as the matrix material.

31. The production method according to claim 29, wherein the mixing step uses gingival fibroblasts as the cells and one or more components selected from at least (b) collagen and derivatives thereof as the matrix material.

32. The production method according to claim 29, wherein the mixing step uses as the matrix material at least platelet-rich plasma for (d) plasma and platelets.

33. A method of evaluating a composition for regenerating periodontal soft tissue, comprising steps of:
preparing a test composition for regenerating periodontal soft tissue, said test composition comprising cells selected from mesenchymal stem cells and gingival fibroblasts, and a matrix material;
subcutaneously injecting the test composition for regenerating periodontal soft tissue in a dorsal region of a rodent and obtaining a ratio for the height of an elevated region thus formed at a predetermined time after injection to the height of the elevated region at the time of injection, and/or a ratio for the volume of the elevated region thus formed at a specified time after injection to the volume of the elevated region at the time of injection; and
evaluating the capacity to regenerate the free gingiva based on the obtained height ratio and/or volume ratio.

34. A method of producing a composition for regenerating periodontal soft tissue, comprising steps of:
preparing a test composition for regenerating periodontal soft tissue, said test composition comprising cells selected from mesenchymal stem cells and gingival fibroblasts, and a matrix material;
subcutaneously injecting the test composition for regenerating periodontal soft tissue in a dorsal region of a rodent and obtaining a ratio for the height of an elevated region thus formed at a specified time after injection to the height of the elevated region at the time of injection, and/or a ratio for the volume of the elevated region thus formed at a specified time after injection to the volume of the elevated region at the time of injection;
evaluating the capacity to regenerate the free gingiva based on the obtained height ratio and/or volume ratio; and
determining, based on the free gingival regeneration capacity, components for the composition for regenerating periodontal soft tissue and producing the composition for regenerating periodontal soft tissue based on the determined composition.
